(19) United States
(12) Patent Application Publication
(10) Pub. No.: US 2022/0265706 A1
(43) Pub. Date: Aug. 25, 2022

(54) PHARMACEUTICAL COMPOSITION CONTAINING COMPLEXES OF ALLOFERON AND ZINC

(71) Applicants: German Petrovich Bekker, Moscow (RU); Oleg Vladimirovich MATUSEVICH, Sankt-Peterburg (RU); Boris Alekseevich NIKONOV, St.Peterburg (RU)

(72) Inventors: German Petrovich Bekker, Moscow (RU); Oleg Vladimirovich MATUSEVICH, Sankt-Peterburg (RU); Boris Alekseevich NIKONOV, St.Peterburg (RU)

(73) Assignee: German Petrovich Bekker, Moscow (RU)

(21) Appl. No.: 17/627,741

(22) PCT Filed: Dec. 11, 2020
(86) PCT No.: PCT/RU2020/000622

§ 371 Date: Jan. 17, 2022

(30) Foreign Application Priority Data
Dec. 29, 2019 (RU) 2019144881

(51) Int. Cl. A61K 33/30; A61K 38/10; A61P 31/12; C07K 7/08

(52) U.S. Cl. CPC A61K 33/30; C07K 7/08; A61P 31/12; A61K 38/10

(57) ABSTRACT

A composition containing biologically active complexes of zinc and the oligopeptide alloferon. A pharmaceutical composition with antiviral and immunomodulatory activity, the active ingredient is a mixture of biologically active complex compounds formed by the reaction of the oligopeptide alloferon with zinc ions in an aqueous medium, zinc and alloferon in solution form complex compounds having different compositions, and the concentration of the solutions of alloferon and a zinc salt is selected that the molar ratio of the components alloferon and zinc salt is from 1:1 to 1:2. A method for producing a pharmaceutical composition having zinc salt and alloferon dissolved separately in equal volumes of 0.9% solutions of sodium chloride or sodium acetate, mixed and pH is adjusted to 6.5-8.0. The composition, the contents and properties of which make it possible to achieve in solution a high concentration of complex compounds formed by the reaction of alloferon with zinc ions.

### CROSS REFERENCE TO RELATED APPLICATION

This application is a national stage entry of PCT/RU2020/000622 filed Dec. 11, 2020, under the International Convention and claiming priority over Russian Patent Application No. RU2019144881 filed Dec. 29, 2019.

### FIELD OF THE INVENTION

The invention is directed to pharmaceuticals and relates to optimizing the makeup of a composition comprising a bioactive zinc-alloferon peptide complex.

### BACKGROUND OF THE INVENTION

One known Patent RU2470031 (Kiselev, O. I., Yershov, F. I., published 20 Dec. 2012.) describes zinc-containing peptide complexes with antiviral and immunomodulatory activities.

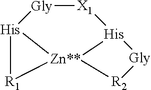

The above solution has been chosen as the closest prior art.

This type of complex peptide compounds with zinc are the substance of Patent RU2470031. However, the structural formula as proposed by the above patent inventors for such complexes is based solely on general details of zinc and histidine-containing peptide properties and on computer simulation data, thus being merely theoretical. The complexes claimed by the inventors pertain to a wide range of peptides with molecules containing various (but at least three) histidine residues.

The primary subject considered by the inventors is alloferon oligopeptide (Alloferon 1 under Patent RU2172322, Alloferons-immunomodulatory peptides, Chernysh, S. I., Kim Soo In, Bekker, G. P. et al.). However, the inventors extended their conclusions not only to the broad alloferon family, but also to other peptides enriched with histidine. The inventors of the above patent assumed that histidine-containing peptides form chelates with zinc, wherein zinc forms coordinate bonds with histidine residues. However, the inventors did not provide any experimental data to substantiate the complexes' structural formula which is the essence of the invention. Without such substantiation, the validity of the structural formula is largely open to doubt. Also open to doubt is the fact that the complexes' formula and the conclusions about their biological activity are a priori extended to a wide range of peptides that were not studied.

From the above patent it follows that aqueous solutions were used to assess the complexes' biological properties. It is, however, known that complex compounds demonstrate complex behavior in aqueous media (O. V. Sergeeva, Reaktsii v vodnykh rastvorakh. Slozhnye ionnye ravnovesia (Reactions in Aqueous Solutions. Complex Ion Equilibriums). Minsk, 2009). In view of this, no firm conclusions can be drawn with respect to the complexes' structure or makeup merely on theoretical assumptions. Complex compounds are substances whose structure is essentially instable (Grinberg, A. A., Vvedeniye v khimiyu kompleksnykh soedineniy (An Introduction into the Chemistry of Complex Compounds), 4^{th} Edition, L., 1971). In aqueous solutions, they are always a mixture of various forms, the equilibrium between which is defined by stability (instability) constants. In case of complexes comprising peptides or proteins, i.e. soft ligands, this is even more pronounced (Neorganicheskaya biokhimiya (Inorganic Biochemistry), Mir, M., 1978, Vol. 1, p. 152-162, Section: Metal Amino Acid and Metal Peptide Complexes). In case of an alloferon-zinc system, this is magnified by the facts that: firstly, zinc is a weak complexing agent, and, secondly, alloferon comprises, in addition to histidine, other functional groups with coordinate bonding ability.

The inventors of RU2470031 have demonstrated that the biological activity of alloferon-zinc complexes exceed that of the original peptide. However, while providing data on the biological properties of the complexes, the inventors of the above patent have not provided any details of a method for producing thereof. The specification does not contain any details on the zinc salts used to produce the complexes, or discloses a method for producing thereof, or allowable conditions (pH, solvents, ionic strength, additional components) under which such complexes exist.

Patent RU2470031 is, therefore, merely theoretical and, as such, is neither reproducible, nor practically useful.

For practical purposes, it is necessary to establish, in the first place, whether any alloferon-zinc complexes are present in an aqueous solution, and, in the second place, find conditions of their existence.

Complex compounds result from interactions between substances, one of which has electron acceptor properties, i.e. is a complexing agent, and the other is an electron donor, i.e. a ligand. By mixing two solutions containing metal (complexing agent) ions and a ligand (herein, the ligand is alloferon), a complexing, i.e. ligand to metal bonding, process is initiated. The ligand binds to the metal in a staged manner until the ligand amount becomes equal to the complexing agent coordination number (in case of zinc, it is generally 4). A dynamic equilibrium is established, at which both complexing and decomplexation processes occur. The solution, therefore, simultaneously contains all forms of complexes formed at various complexing stages and the initial substances. Their proportion depends on the strength of the bonds so formed (Grinberg, A. A., Vvedeniye v khimiyu kompleksnykh soedineniy (An Introduction into the Chemistry of Complex Compounds)), 4^{th} Edition, L., 1971).

Complex stability is determined both by fundamental factors (the natures of the complexing agent and the ligands), and external conditions (temperature, solvent nature, ionic strength, solution makeup). Regarding the fundamental factors affecting the complex stability, special mention should be made of the central atom and the ligand natures, the ligand structure, and steric factors. Ligands having several binding sites (polydentate ligands) may form cyclic complexes, i.e. chelates, of higher stability.

Peptides as ligands have several metal binding sites, i.e. they are polydentate ligands. Such sites may include: terminal amino and carboxy groups, amino nitrogen atom of the peptide bond, as well as side functional groups of amino-acid residues. As such, peptides typically form chelate-type complexes.

Alloferon, in addition to the above active groups, comprises 4 residues of histidine, i.e. an amino acid comprising an imidazole ring prone to complexing. This confers to alloferon the properties of an active ligand. This aspect is used in the above Patent RU2470031. As the coordination number of zinc is typically 4, and an alloferon molecule contains 4 histidine residues, the chelate complex formula proposed by the inventors of Patent RU2470031 appears to be obvious. However, in view of the presence, in the alloferon molecule, of other functional groups capable of forming coordinate bonds with metal ions, other structures may also exist. Furthermore, particular features of complexing in an aqueous medium do necessarily affect the makeup and structure of resulting compounds.

Complex compounds formed by alloferon and alloferon derivatives with copper ions in aqueous solutions have been thoroughly studied by a group of Polish scientists and described in a number of publications. In a paper by T. Kowalik-Jankowsca, L. Biega, M. Kuzer, D. Konopinska. Mononuclear Copper (II) complexes of alloferon 1 and 2: A combined potentiometric and spectroscopic studies. J. Inorg. Biochem. 103, 2009, 135-142, the authors provide a detailed analysis of the equilibrium in an alloferon-copper system (at their equimolecular proportions) based on potentiometric titration and spectrometry data. Copper and zinc, as complexing agents, have similar properties, so the data obtained for copper may be taken into account in studying zinc-alloferon complexes.

The works by the Polish scientists have demonstrated that, in an alloferon-copper system in aqueous solutions, many chelate-type compounds are forming, whose makeup and amount largely depend on the pH of the medium. It has been demonstrated that, at all pH values, copper ions and alloferon form complexes in equilibrium with the initial components. It has been found that a copper ion binds with histidine residues and necessarily with an alloferon terminal amino group. The number of copper bonds with histidine nitrogen depends on the pH of the medium and increases with increasing pH. It may be bound with one, two or three histidine residues. It is important that one of the four histidine residues always remains free and not bound with a metal ion. Such structure of the complexes is different from the formula disclosed in Patent RU2470031, wherein binding to 4 histidine residues is considered. The authors of the Polish publication have demonstrated that binding to 4 histidine residues is possible, but only where a terminal amino group is blocked, such as by acetylation.

An important factor discovered by the authors of the above publication is that, in an alloferon-copper system, complex compounds of various makeups and structures are present simultaneously. Thus, polynuclear complexes were found in it, in which complexes several metal ions were coordinated with one alloferon molecule. As such, an alloferon-copper system in an aqueous solution has complex makeup, wherein both complex compounds of various makeups and the initial substances, i.e. alloferon and a copper salt, are present simultaneously.

Similar patterns should occur in alloferon-zinc system aqueous solutions. However, it is important to note that zinc's complexing ability, in the Irving-Williams Series, is less than that of copper (O. V. Sergeeva, Reaktsii v vodnykh rastvorakh. Slozhnye ionnye ravnovesia (Reactions in Aqueous Solutions. Complex Ion Equilibriums). Minsk, 2009). As such, zinc complex compounds are typically less stable than those of copper. However, presumably, the nature and patterns of complex compound existence in an alloferon-zinc system may be close to those described above for copper.

### SUMMARY OF THE INVENTION

It is, therefore, an objective of the present invention to provide a pharmaceutical composition with enhanced biological activity, the composition comprising: active alloferon-zinc complexes with an optimal makeup, and conditions that allow producing a solution with a maximum concentration of such compounds.

A technical result of the invention is a composition with a makeup and properties allowing achievement, in a solution, of a high concentration of complex compounds formed though alloferon interaction with zinc ions, and with a biological activity, wherein the antiviral activity of the composition is higher than the antiviral activity of alloferon itself.

The above technical result is attained by the claimed pharmaceutical composition with antiviral and immunomodulatory activities, the composition comprising, as active ingredients, alloferon and zinc, characterized in that its active principle is a mixture of bioactive complex compounds formed through interaction of alloferon oligopeptide with zinc ions in an aqueous medium, wherein zinc and alloferon, in a solution, form complex compounds of various makeups, and wherein alloferon and zinc salt solution concentration is selected such that the molar ratio between alloferon components and zinc salt is from 1:1 to 1:2.

It is allowable that the composition is made up by alloferon and a zinc salt used in an equimolecular or larger amount; 0.9% sodium chloride or sodium acetate solution is used as a medium, and the solution pH is within OT 6.5 to 8.0 (preferably 7.5).

It is allowable that zinc acetate or chloride is used as the zinc salt.

It is allowable that the composition is made by first making a first solution which is produced by dissolving 100 mg of alloferon in 50 ml of 0.9% sodium chloride or sodium acetate solution, simultaneously making a second solution which is produced by dissolving 17.4 mg of zinc acetate dihydrate or 10.8 mg of anhydrous zinc chloride in 50 ml of 0.9% sodium chloride or sodium acetate solution; then adding, gradually while stirring, the second solution to the first solution; 30 minutes thereafter, the solution pH is checked and adjusted to a value of 6.5-8.0 with an 0.1 M sodium hydroxide or chlorohydric acid solution; the resulting solution is sterilized by filtering through a 0.2 μm pore size filter, metered into ampoules or vials which are then tightly sealed.

It is allowable that the final concentrations of the composition components are provided as follows: 1 mg/mL of alloferon, zinc salt: 0.052 to 0.104 mg of zinc per mL.

It is allowable that the composition is made by first making a first solution which is produced by dissolving 100 mg of alloferon in 50 ml of 0.9% sodium chloride or sodium acetate solution, simultaneously making a second solution which is produced by dissolving 34.8 mg of zinc acetate dihydrate or 21.6 mg of anhydrous zinc chloride in 50 ml of 0.9% sodium chloride or sodium acetate solution; then adding, gradually while stirring, the second solution to the first solution; 30 minutes thereafter, the solution pH is checked and adjusted to a value of 6.5-8.0 with an 0.1 M sodium hydroxide or chlorohydric acid solution; the resulting solution is sterilized by filtering through a 0.2 μm pore size filter, metered into ampoules or vials which are then tightly sealed.

### BRIEF DESCRIPTION OF THE DRAWINGS

FIG. 1 shows a mass spectrum of 0.001 M alloferon aqueous solution.

FIG. 2 shows a mass spectrum of 0.001 M alloferon-zinc complex aqueous solution at a molar ratio between alloferon and zinc of 1:1.

FIG. 3 shows a mass spectrum of 0.001 M alloferon-zinc complex aqueous solution at an alloferon to zinc ratio of 1:2.

FIG. 4 shows a fragment of a mass spectrum of an alloferon-zinc complex. Triple-charged ion m/z 443.1721. Experimental data.

FIG. 5 shows a fragment of a theoretically derived mass spectrum of an alloferon-zinc complex, m/z 443.1721.

FIG. 6 shows a mass spectrum of an alloferon+zinc complex in a 0.9% sodium chloride solution.

FIG. 7 shows a mass spectrum of 0.001 M solution of an alloferon-zinc complex at the pH of 4.0.

FIG. 8 shows a mass spectrum of 0.001 M solution of an alloferon-zinc complex at the pH of 6.0.

FIG. 9 shows a mass spectrum of 0.001 M solution of an alloferon-zinc complex at the pH of 8.0.

FIG. 10 shows solution titration curves.

FIG. 11 shows differential solution titration curves.

FIG. 12 shows differential solution titration curves.

### DETAILED DESCRIPTION

The development has been made using zinc acetate and zinc chloride as the zinc salts. These are the only zinc salts included into pharmaceuticals for parenteral administration, primarily into various forms of insulin. As a solvent, 0.9% sodium chloride and sodium acetate aqueous solutions were used. Herein, no other isotonic solutions (Ringer's solution, etc.) may be used, as they contain additional calcium ions forming insoluble compounds with zinc.

The alloferon and zinc salt containing solutions were studied by the potentiometric titration and mass spectrometry methods.

It has been found that a complexing process occurs in an alloferon-zinc system in an aqueous medium. However, the solution always contains an equilibrium mixture of the initial components, i.e. alloferon and zinc salt, and complex compounds formed by them. It has been found that, in a solution, zinc and alloferon form complex compounds of various makeups.

Complexes that are mainly formed are those in which an alloferon molecule is bound with one zinc ion. The solution, however, also contains complexes in which an alloferon molecule is bound with two zinc ions, as well as dimers in which one zinc ion is bound with two alloferon molecules. An alloferon-zinc system in an aqueous solution, therefore, contains a complex mixture of substances including the initial components and complexes of various structures and makeups. It is, however, obvious that such complexes differ, in their makeup and structure, from the formula disclosed in Patent RU2470031.

It has been found that the makeup and concentration of the complexes so formed depend on the pH, alloferon to zinc ratio, and ionic strength of the solution. Concentration of the complexes increases with increasing pH. The same effect is produced with a zinc salt amount above the equimolecular value. By using 0.9% sodium chloride or sodium acetate solutions with a pH value from 6.5 to 8.0 as the medium, a composition could be obtained with a complexing rate close to quantitative complexing.

As a result, the developed composition comprises alloferon-zinc complex compounds of various structures, initial alloferon and zinc salt (acetate or chloride), 0.9% sodium chloride or sodium acetate solution and has a pH in the range from 6.5 to 8.0, preferably 7.5. The composition is produced by using alloferon and an equal or larger amount of zinc salt. The composition is produced by mixing equal volumes of zinc acetate or chloride and alloferon solutions a 0.9% sodium chloride or sodium acetate solution, followed by adjusting its pH to said value of 6.5-8.0 (preferably 7.5).

Biological activity of the claimed compositions has been demonstrated experimentally. Allokin-alfa, Lyophilisate for Solution for Subcutaneous Administration, containing 1 mg of pure alloferon without excipients and a conventional antiherpetic preparation were used as reference products. Antiviral activity of the developed composition, previously demonstrated with respect to an influenza virus (Patent RU2470031), has been tested with respect to a herpes virus. As a result of the tests, the data of Patent RU2470031 were confirmed and an antiviral activity higher than that of alloferon was demonstrated. Immunomodulatory properties of the developed composition, demonstrated through the example of interferon gamma and Interleukin 18 induction, was also higher than that of pure alloferon peptide.

The above conclusions are supported by the following examples.

### Example 1. Mass Spectrometry Studies

Under the guidelines applicable to the equipment, it is undesirable for ESI-MS mass spectrometry solutions to contain inorganic salts, so the majority of the solutions were produced on the basis of deionized water. Solutions with various pH values were produced by using a formate buffer (pH 4 and 6) and a Tris buffer (pH 8.0). Alloferon and zinc salt solution concentration was 0.001 M, molar ratios between the components were 1:1 and 1:2. Zinc acetate was used as the zinc salt.

The work was done using LTQ OrbiTrap Velos Mass Spectrometer with electrospray ionization at atmospheric pressure and Xcalibur control and data processing software.

Mass spectrometer operating conditions:

Drying gas flow: 10 conventional units

Auxiliary gas flow: 5 conventional units

Drying gas temperature: 80° C.;

Auxiliary gas temperature: 280° C.;

Capillary voltage: 3500V;

Direct introduction of a sample with a 500 mcL syringe at the flow rate of 5 mcL/min;

Detection in the total ion current mode (SCAN): ion monitoring in the m/z range from 300 to 1500 (positive ionization).

Monoisotopic masses of certain possible alloferon ions are given below:

Alloferon: Gross formula C₅₂H₇₆N₂₂O₁₆, monoisotopic molecular weight M. W.=1264.58096

Singly charged ion m/z [M+H]⁺=1265.58824

Double-charged ion m/z [M+2H]²⁺=633.29776

Double-charged ion with sodium m/z [M+2Na]²⁺=644.28873

Triple-charged ion m/z [M+3H]³⁺=422.53426

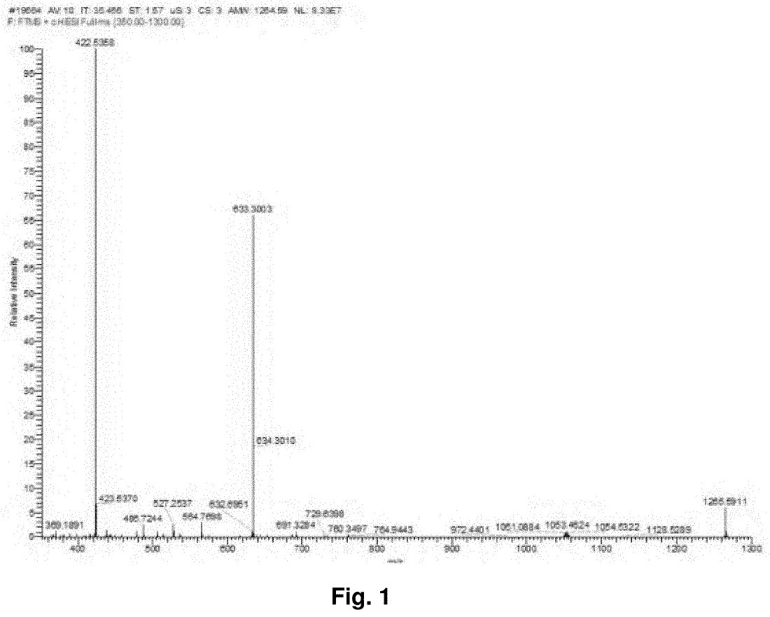

FIG. 1 shows a mass spectrum of 0.001 M alloferon aqueous solution. Well distinguished are several ions that are characteristic of a peptide. Ion with m/z 1265.5896 is a singly charged ion, ion with m/z 633.2992 is a double-charged ion [M+2H]²⁺, and a triple-charged ion [M+3H]³⁺ with m/z 422.5351.

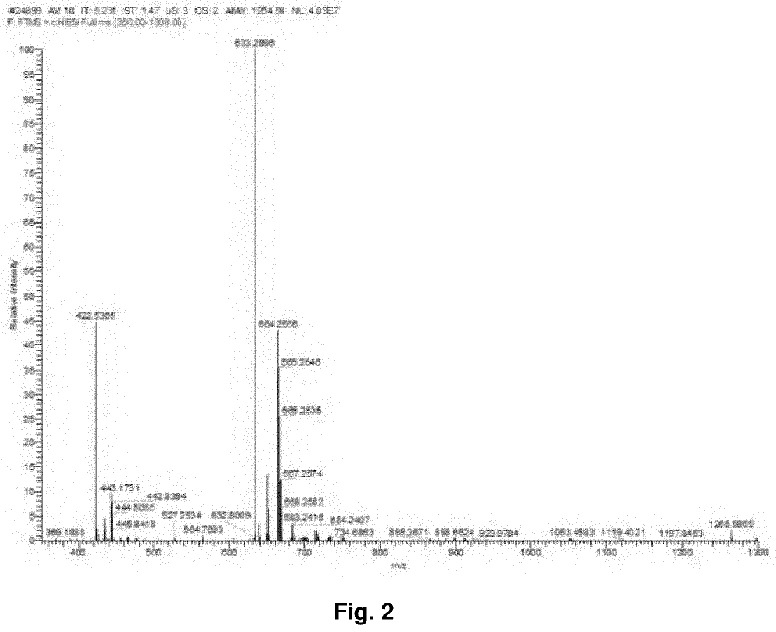

FIG. 2 shows a mass spectrum of a 0.001 M alloferon and zinc salt containing solution with a molar ratio between alloferon and zinc of 1:1. From comparison of spectra of FIG. 1 (alloferon) and FIG. 2 (the composition) it can be seen that signals of new ions have appeared in the system, which may be explained only by formation of new alloferon-zinc complex compounds. Low intensity of the new ion signals is indicative of that the free alloferon content in the solution is much higher than the complex concentration.

The spectrum of FIG. 2 includes a signal with m/z 443.1731 corresponding to a triple-charged ion of the zinc complex [M+Zn+H]³⁺, as well as a signal with m/z 664.2556 corresponding to a double-charged ion of the [M+Zn]²⁺ complex. Both signals correspond to a complex with a C₅₂H₇₆N₂₂O₁₆Zn gross formula.

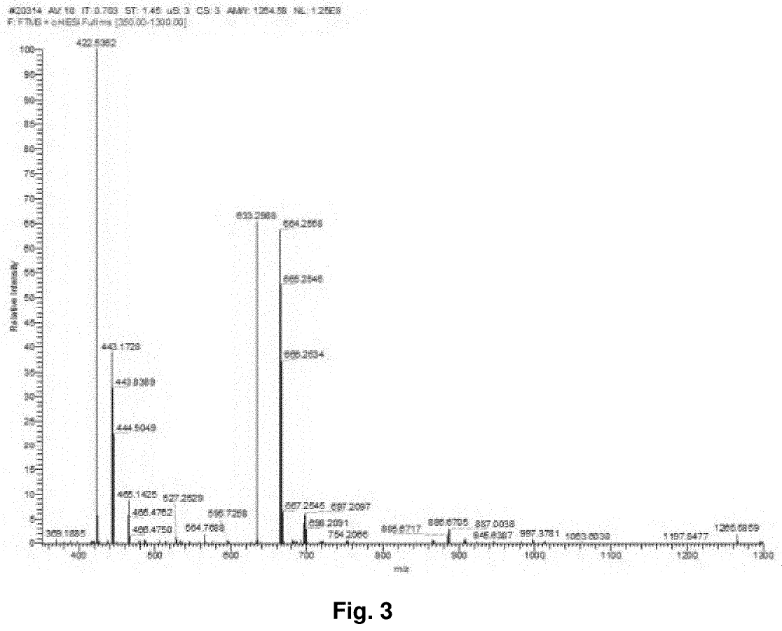

FIG. 3 shows a mass spectrum of 0.001 M alloferon-zinc complex aqueous solution with a molar ratio between alloferon and zinc of 1:2. It demonstrates that, with increasing zinc content, the complex concentration in the solution significantly increases. No compositions with alloferon to zinc ratios higher than 1:2 were studied.

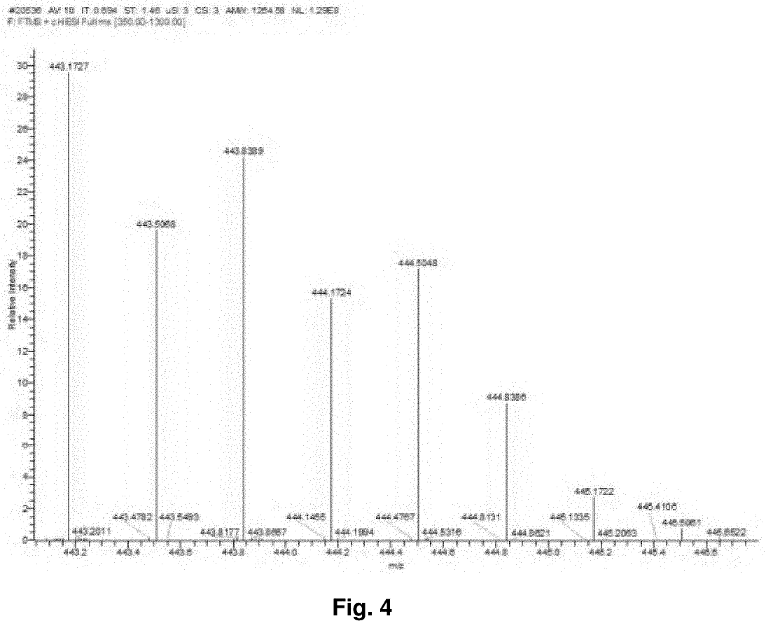

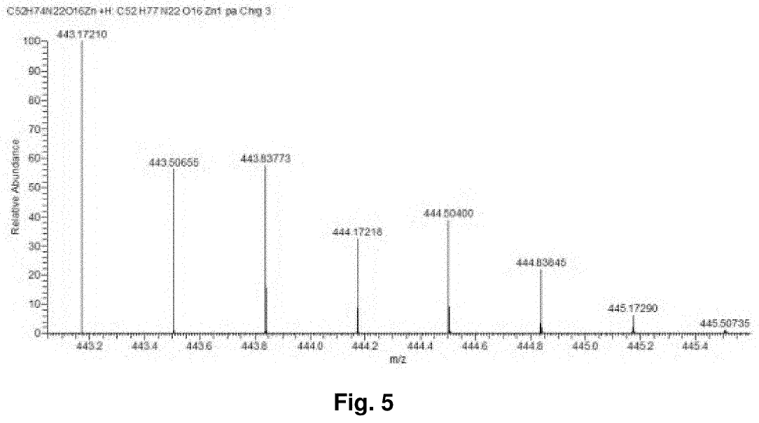

As shown in FIG. 4 and FIG. 5, the actual and theoretical mass spectra of a triple-charged ion of the complex are substantially identical, which is indicative of that this signal in the mass spectrum is correctly interpreted as corresponding to a complex with a C₅₂H₇₆N₂₂O₁₆Zn gross formula. The same agreement between the theoretical and the actual data was obtained for a double-charged ion.

The spectra further include signals with m/z 465.1425 and 697.2097. They correspond to a complex with a C₅₂H₇₂N₂₂O₁₆Zn₂ gross formula, wherein alloferon is bound with 2 zinc atoms simultaneously. A comparison made between the actual and a theoretically derived mass spectrum of a triple-charged ion with m/z 465.1425 confirms such identification. Furthermore, a triple-charged ion with m/z 886.6705 has been observed, which corresponds to an alloferon-zinc dimer C₁₀₄H₁₅₂N₄₄O₃₂Zn.

The mass spectrometry study, therefore, demonstrates that, in an aqueous medium, alloferon in combination with zinc salts forms complex compounds of various shapes and makeups.

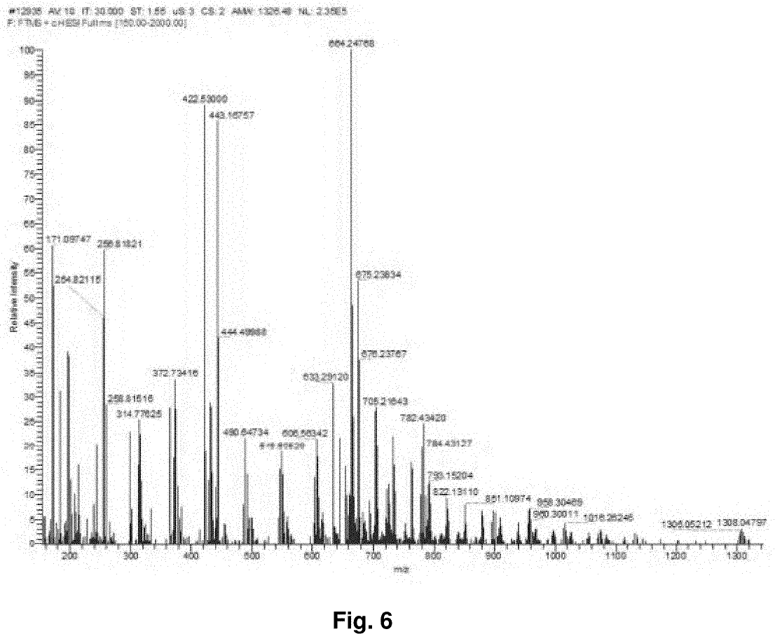

Despite the existing technical limitations, mass spectra for an alloferon-zinc system in saline solutions were obtained. FIG. 6 shows a mass spectrum of the composition in a 0.9% sodium chloride solution. Intensities of the double- and triple-charged signals with m/z 664.2558 and 443.1728 are indicative of a high content of alloferon-zinc complexes C₅₂H₇₆N₂₂O₁₆Zn achieved in an isotonic solution. The intensity of double-charged ion signals of a complex with m/z 664.2558 in the spectrum of FIG. 6 is significantly higher than that of an alloferon ion m/z 633.29776, a triple-charged ion with m/z 443.1721 at its level (m/z 422.53426). The mass spectrum of the composition in a sodium acetate solution is similar to that of FIG. 6.

Content of complex compounds in an alloferon and zinc solution was studied as a function of the solution pH value.

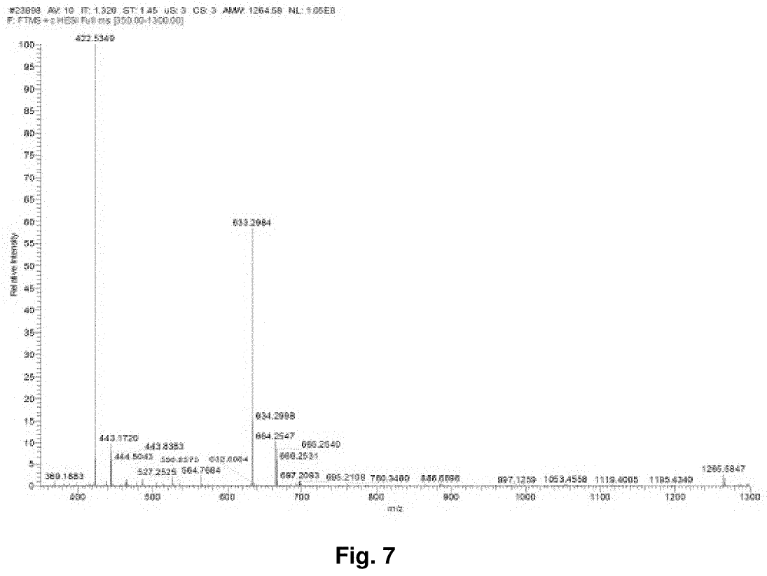

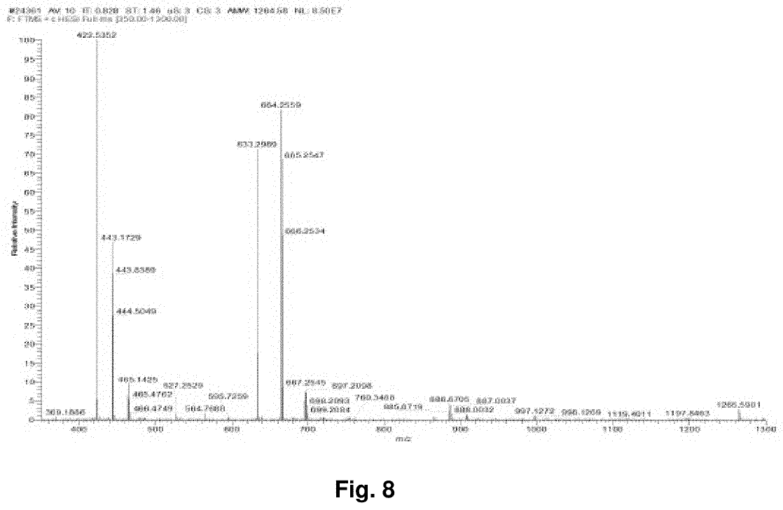

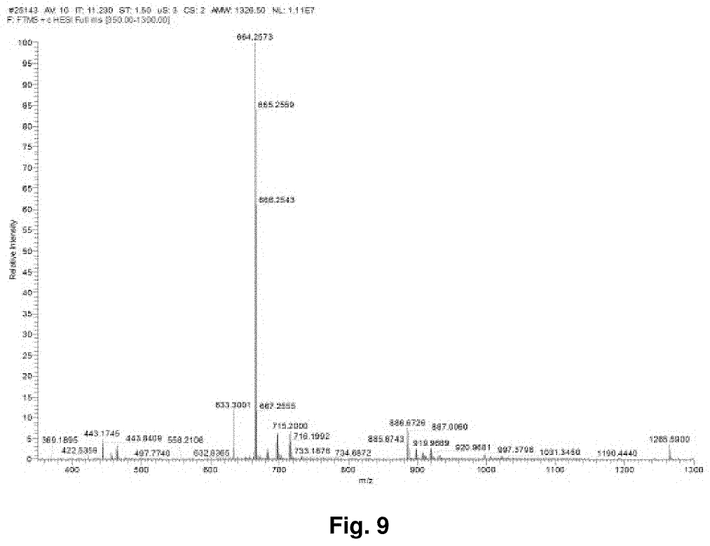

The results (FIG. 7-9) demonstrate that alloferon-zinc complex compound content depends on the pH value. With the pH value increasing within the range from 4 to 8, an increase in intensities of respective signals in the mass spectra of zinc-alloferon complex compound solutions is observed, which may be indicative of an increase in their concentration in the solution due to a change in pH.

The maximum signal intensity for a double-charged zinc complex with m/z 664.2558 is observed at the pH of 8. However, there is not any signal of triple-charged ion of the complex at this pH value.

The above mass spectrometry results most probably indicate that:

alloferon with zinc salts in an aqueous solution form complex compound;

the aqueous solution simultaneously contains alloferon-zinc complexes of various makeups and structure and free alloferon;

concentration of the complexes depends on the solution pH, i.e. with increasing pH, the concentration increases to reach its maximum at the pH of 8.0;

concentration of the complexes depends on the alloferon to zinc ratio and ionic strength of the solution.

Conclusion: a composition with the maximum concentration of complex compounds comprises alloferon, a zinc salt in an amount equal to or higher than the equimolecular amount in a 0.9% sodium chloride or sodium acetate solution with a pH of 6.0 to 8.0.

### Example 2. Potentiometric Titration

The subject solutions had the alloferon and zinc concentration of 0.001 M at the molar ratio of 1:1. Solvent: 0.9% sodium chloride solution. The volume of a solution to be titrated was 30 mL. The solutions were produced by using zinc acetate dihydrate Zn(CH₃COO)₂*2H₂O and anhydrous zinc chloride ZnCl₂.

The work was done using ATP-02 Automatic Potentiometric Titrator capable of producing integrated and differential titration curves.

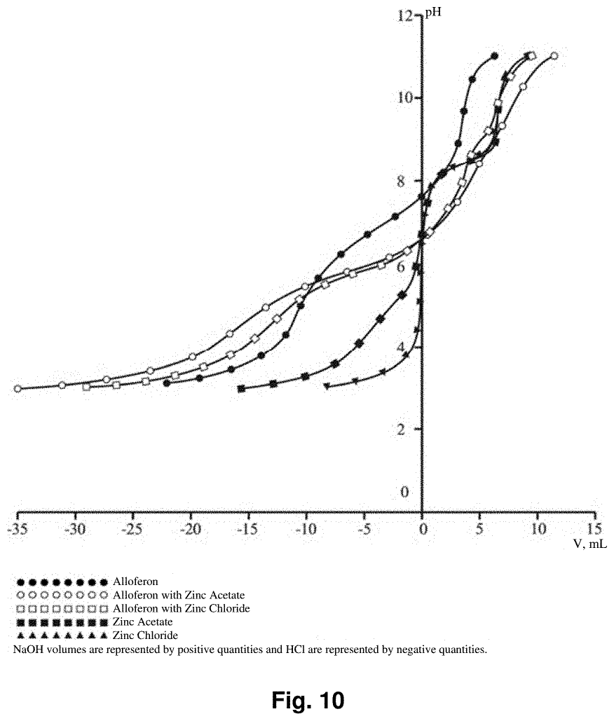

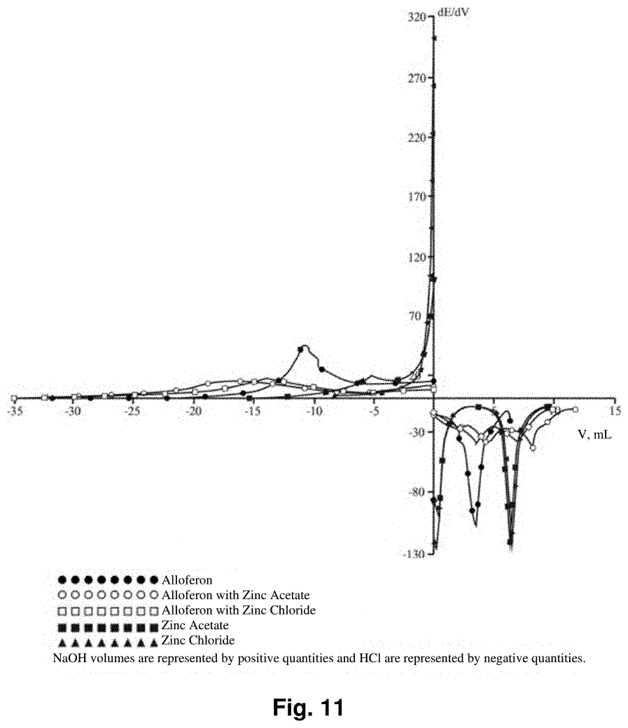

Titrations were done both for a composition of alloferon with zinc salts and for solutions of initial components: alloferon, zinc acetate and zinc chloride. Titrations were carried out separately in the acid and alkali regions. Each titration was performed three times. Averaged titration data are given in the integrated (FIG. 10) and differential (FIG. 11) forms.

The alloferon titration curve includes two well pronounced jumps at the pH of 4.75 and 9.4, which, quantitatively, strictly correspond to the presence of 4 histidine residues and one terminal COOH group.

Alloferon+zinc salt mixture titration curves are substantially different. Firstly, by adding a zinc salt into an alloferon solution, the solution pH value is significantly (from 7.5 to 6.5) changed. This very fact allows suggesting that the components in the subject system interact with each other.

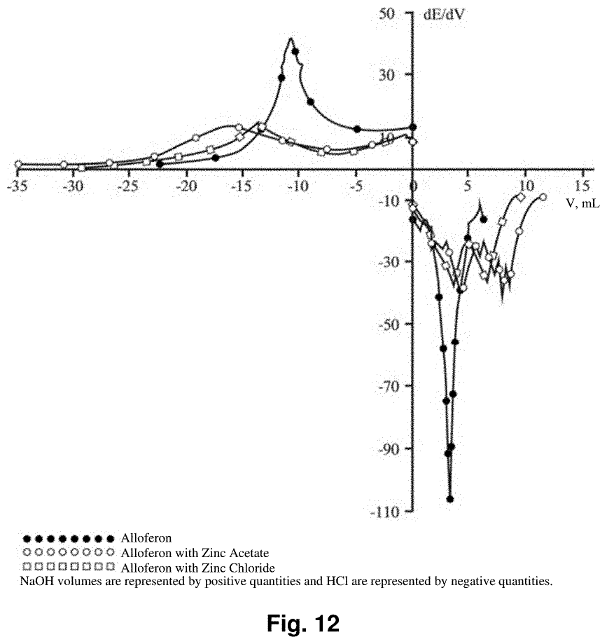

Secondly, jumps in the composition solution titration curves as compared with the curve for alloferon are very diffuse (the amplitude of the dE/dV jump in the differential curves is dramatically decreased). Thirdly, the titration curves do not include any new jumps. These differences may not be described by merely adding pure alloferon titration curves to pure zinc salt solution titration curves (FIG. 10). The differential curves (FIG. 11 and, in more detail, FIG. 12) demonstrate it more clearly. These facts make it clear that there is an interaction between alloferon and the zinc salts. The only possible interaction is complexing. However, the low amplitude of the titration jumps is indicative of a weak bond between alloferon and zinc.

In the acid region, no jump at the pH of 4.75 characteristic of alloferon is observed, while a new jump at the pH of 4.25 appears. There is also a new, but very weak, jump at the pH of 6.5, which is more pronounced in case of zinc acetate.

The most important changes are observed in the alkali region. Thus, there is eventually no jump characteristic of either alloferon or zinc salts at the pH of 9.4. This is indicative of a low content of free alloferon and zinc ions in the alkali region. They are, therefore, mainly included in a complex compound. However, there are two new jumps: one at the pH of 8.0 and the other at about 10.0, which may be interpreted as titration jumps corresponding to different forms of the complex. In the alkali region, complexes containing deprotonated forms of alloferon may exist at high pH values (above 8.0).

Of the most interest is a narrow region between the jumps at the pH of 6.5 and 8.0. Therein, an uncharged alloferon molecule may interact with zinc salts to form a chelate-type structure.

The data so obtained indicate that:

alloferon and a zinc salt interact with each other in a solution;

the interaction between alloferon and the zinc salt to form complex compounds is observed throughout the pH range from 4.0 to 10.0;

the pH region from 6.5 to 8.0 is preferable for pharmaceutical purposes;

the potentiometric titration data confirms the patterns found by the mass spectrometry studies (Example 1) of the alloferon-zinc system.

Conclusion: alloferon and zinc in a 0.9% sodium chloride solution with a pH from 6.5 to 8.0 form a composition with a maximum concentration of complex compounds.

### Example 3. Producing the Compositions

Composition 1 (molar ratio 1:1).

100 mg of alloferon is dissolved in 50 ml of 0.9% sodium chloride or sodium acetate solution—Solution 1. Simultaneously, 17.4 mg of zinc acetate dihydrate (or 10.8 mg of anhydrous zinc chloride) is dissolved in 50 ml of 0.9% sodium chloride or sodium acetate solution—Solution 2. Solution 2 is added, gradually while stirring, into Solution 1.30 minutes thereafter, the solution pH is checked and adjusted to a value of 7.5 with an 0.1 M sodium hydroxide or chlorohydric acid solution. The resulting solution is sterilized by filtering through a 0.2 μm pore size filter, metered into ampoules which are then vacuum sealed or vials which are then tightly sealed with plugs and caps.

Saline solutions for producing the compositions are produced by dissolving 9.0 g of sodium chloride or 14.75 g of sodium acetate trihydrate in 1 liter of purified water. The solutions are sterilized by autoclaving and kept at a temperature of +4-8° C. before use.

Composition 2 (molar ratio 1:2).

The production process is similar to that for Composition 1, except that 34.8 mg of zinc acetate dihydrate (or 21.6 mg of anhydrous zinc chloride) is used.

### Example 4. Interferon Gamma (IFN-γ) and Interleukin 18 (IL-18) Induction

To assess the immunomodulatory activity (IA) of the claimed composition and to discover the contribution from the complex compounds contained therein, a study has been performed to determine the level of cytokine (IFN-γ and IL-18) induction both by the compositions and the initial components, i.e. alloferon and a zinc salt (acetate). Furthermore, cytokine induction caused by using both components (alloferon and the zinc salt), where administered to a body separately in time, were determined. Six samples were tested:
1. Alloferon (Allokin-alfa, Lyophilisate for Solution for Subcutaneous Administration)—a reference product,
2. Composition 1,
3. Composition 2,
4. Zinc acetate
5. Alloferon and zinc acetate administered separately, one 15 minutes after the other,
6. 0.9% sodium chloride solution—control.

Samples 1, 2, 3, and 5 contained alloferon at the concentration of 1 mg/mL. The alloferon solution in Samples 1 and 5 was produced by dissolving the contents of an Allokin-alfa ampoule (1 mg of alloferon) in 1 mL of 0.9% sodium chloride solution. The zinc acetate solution in Examples 4 and 5 had the concentration of 0.17 mg/mL (corresponding to the concentration in Composition 1). To determine the background content of IFN-γ and IL-18 in the subject animals' blood serum, a 0.9% sodium chloride solution was used.

The experiments enrolled BALB/C mice, females, weighing 16 to 22 grams, 18 weeks of age, divided into 6 groups of 15 individuals each. The samples were administered subcutaneously in a single dose of 1 mg of alloferon per 1 kg laboratory animal body weight. Sample 5 components were administered separately, one 15 minutes after the other. The amount of administered zinc acetate (Samples 4 and 5) corresponded to its amount in Composition 1.

The interferon gamma level in the blood serum was determined by the solid phase heterogeneous immunoassay method with the use of specific antibodies 3 hours after the sample administration, i.e. when the maximum level of IFN-γ induction is observed.

The Interleukin 18 induction level was determined by using the procedure described in Patent RU 2482866; based on the data from that document, the point in time 36 hours after the sample administration was selected to determine the IL-18 level. In case of the control group, the confidence interval was calculated based on measurements of the same sample dilutions. For the experimental groups, the confidence interval was calculated based on three samples from three animals for each measuring point. Testing results for the subject samples are given in Table 1.

TABLE 1
Interferon Gamma and Interleukin 18 Concentrations in Blood Serum

| | | Interferon Gamma, | Interleukin 18, |
|---|---|---|---|
| | | after 3 hours | after 36 hours |
| # | Sample | (mg/mL of blood serum) | (mg/mL of blood serum) |
| 1 | Alloferon | 95 ± 16 | 360 ± 20 |
| 2 | Composition 1 | 140 ± 20 | 402 ± 20 |
| 3 | Composition 2 | 148 ± 20 | 422 ± 25 |
| 4 | Alloferon + zinc | 120 ± 15 | 354 ± 15 |
| | (separate administration) | | |
| 5 | Zinc acetate | 82 ± 8 | 194 ± 12 |
| 6 | 0.9% sodium chloride | 32 ± 8 | 186 ± 12 |
| | solution, control | | |

Based on the results so obtained, the following conclusions can be made.

### Conclusions:

The studied compositions have a specific ability to induce IFN-γ and IL-18.
1. Cytokine induction caused by the compositions containing alloferon-zinc complexes is significantly higher than the cytokine level induced by alloferon.
2. The maximum level of cytokine induction is produced by Composition 2 having the alloferon to zinc ratio of 1:2.
3. The compositions' immunostimulatory activity is not a sum of the initial components' activities, which is indicative of the contribution from the complex compounds formed by them into the immune system activation.

### Example 5. Antiviral Activity of the Compositions Based on a Model of Herpetic Infection in Mice

The developed compositions were assessed for their protective action on the basis of a herpetic infection model. The following reference products were used: 1/Allokin-alfa, Lyophilisate for Solution for Subcutaneous Administration, a medicinal product used to treat genital herpes, containing 1 mg of alloferon per ampoule; 2/Acyclovir, a medicinal product used in the conventional herpes therapy.

White outbred mice (females) weighing 16 to 20 g were received from Rappolovo Nursery (Leningrad Oblast) and maintained on a standard diet under controlled vivarium conditions. The animals for the experimental groups were selected on a random basis. The animals were divided into 5 groups of 20 animals each: four experimental groups in which the animals received injections of Composition 1, Composition 2, Allokin-alfa, and Acyclovir, respectively, and one control group in which the animals received injections of 0.9% sodium chloride solution.

The study was made with the use of Herpes Simplex Virus Type 2 (HSV-2) Strain G (ATCC VR-734). The infection was introduced vaginally. Vaginal epithelium was injured with the use of a vaginal smearing scarifier, after which the animals were infected with the virus at a dose of 1.7×10⁵TCID₅₀ in the amount of 30 mcL.

Allokin-alfa and Compositions 1 and 2 were administered to the animals intraperitoneally at a dose of 1.25 mg/kg once a day for 3 days after the infection introduction. Acyclovir was administered in accordance with the same scheme at a dose of 50 mg/kg. The control group received injections of 0.9% sodium chloride solution.

The animals were followed up for 14 days. Mortality in the control and experimental groups were recorded on a daily basis. Based on the data so obtained, mortality rates M (the ratio of animal deaths over 14 days to the total number of infected animals in a group) and the index of protection IP (the ratio between the difference of the control and experimental groups' mortality rates and the control group lethality rate) were calculated.

M=N₁₄/Nx100%,

Where:

N₁₄ is the number of animal deaths in a group on the 14^{th} day after the infection introduction;

N is the total number of animals in a group;

IP=Mc-Me/Mcx100%,

where Mc and Me are lethality percentages in the experimental and the control groups.

The data obtained from the experiment are given in Table 2.

TABLE 2
Protective Effect of the Subject Products Based
on Model of Herpetic Infection in Mice

| | | Experimental Results | | |
|---|---|---|---|---|
| Product | N | N₁₄ | M (%) | IP (%) |
| Composition 1 | 20 | 3 | 15.0 | 75.0 |
| Composition 2 | 20 | 2 | 10.0 | 83.3 |
| Allokin-alfa | 20 | 4 | 20.0 | 66.7 |
| Acyclovir | 20 | 3 | 15.0 | 75.0 |
| Control | 20 | 12 | 60.0 | — |

### CONCLUSION

The claimed compositions comprising alloferon-zinc complex compounds, wherein the alloferon and zinc salt solution concentration is selected such that the molar ratio between alloferon components and zinc salt is from 1:1 to 1:2, demonstrate high antiviral activity, providing, in case of the herpes virus, a protective action exceeding the effect from the initial alloferon (the Allokin-alfa product).

Protective action of the compositions administered at a dose of 1.25 mg/kg is equal to (Composition 1) or higher (Composition 2) than the efficacy of the conventional antiherpetic preparation Acyclovir at a dose of 50 mg/kg.

1. A pharmaceutical composition with antiviral and immunomodulatory activities comprising:
peptide complexes based on an alloferon-zinc compound having as an active principle is a mixture of bioactive complex compounds formed through interaction of alloferon oligopeptide with zinc ions in 0.9% sodium chloride or sodium acetate solutions, followed by adjusting the pH to a value from 6.5 to 8.0, wherein the alloferon and zinc chloride or zinc acetate concentration is selected such that their molar ratio is from 1:1 to 1:2.

2-5. (canceled)
